(19) United States
(12) Patent Application Publication
(10) Pub. No.: US 2022/0356501 A1
(43) Pub. Date: Nov. 10, 2022

(54) ENGINEERED CELLS FOR INCREASED PRODUCTION OF DIFFICULT-TO-EXPRESS PROTEINS

(71) Applicant: Nikhil Goel, Union City, CA (US)

(72) Inventor: Nikhil Goel, Union City, CA (US)

(21) Appl. No.: 17/740,999

(22) Filed: May 10, 2022

Related U.S. Application Data
(60) Provisional application No. 63/186,756, filed on May 10, 2021.

(51) Int. Cl. C12P 21/00; C12N 5/071; C12N 15/85; C12N 9/10

(52) U.S. Cl. CPC C12P 21/00; C12N 5/0682; C12N 15/85; C12N 9/1088; C12Y 205/01018; C12N 2800/107

(57) ABSTRACT

Described are engineered cells that include genetic alterations leading to up- or down-regulation of certain genes in the cells for improved production of a recombinant protein, especially one that is not easily expressed at high levels in unaltered cell lines. Also provided are methods of preparing and using such cells.

### CROSS REFERENCE TO RELATED APPLICATIONS

This application claims the benefit under 35 U.S.C. § 119(e) of the U.S. Provisional Application Ser. No. 63/186,756, filed May 10, 2021, the content of which is hereby incorporated by reference in its entirety.

### BACKGROUND

Cell lines are frequently used for manufacturing protein therapeutic products. Among all commonly used lines, Chinese hamster ovary (CHO) cells remained as the preferred mammalian cell line for the production of recombinant protein therapeutics. Currently, recombinant protein titers from CHO cell culture have reached the gram per liter range which is a 100-fold improvement over similar processes in the 1980s. The significant improvement of titer can be attributed to progress in establishment of stable and high producing clones as well as optimization of culture process.

To improve protein production, various cell line engineering strategies have been employed focusing on extending the longevity of cell culture, accelerating the specific growth rate and increasing the maximum viable cell density. Also, cell line engineering has been employed to improve the folding, transport and secretion of the recombinant protein. Despite these efforts, however, further improvement is needed for the overall efficiency of protein production, particularly for nonnatural and hard to express proteins.

### SUMMARY

The present disclosure provides engineered cells that include genetic alterations leading to up- or down-regulation of certain genes in the cells for improved production of a recombinant protein, especially one that is not easily expressed at high levels in unaltered cell lines. Also provided are methods of preparing and using such cells.

### BRIEF DESCRIPTION OF THE DRAWINGS

FIG. 1. Fold change in microarray signal for GST related enzymes in SUPERCELL™ in comparison to CHOK1.

FIG. 2. Results of qPCR of 5 different CHO cell lines for GST-related enzymes; GSTT2 (Glutathione-S-transferase theta 2), GSTP2 (Glutathione-S-transferase Pi 2), GSTM1 (Glutathione-S-transferase Mu 1), GSTA3 (Glutathione-S-transferase alpha 3). Housekeeping genes as negative control; Gnb1 (G Protein Subunit Beta 1).

FIG. 3. Results of GST activity assay for CHOK1 and SUPERCELL™

FIG. 4. Results of DTT induced UPR effect on relative expression of Rituximab LC transient expression in SUPERCELL™ and ExpiCHO.

FIG. 5. Protein A titers from cotransfection of chaperones with Tysabri HC and Tysabri LC in ExpiCHO.

### DETAILED DESCRIPTION

### II. Definitions

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polynucleotide” includes a plurality of polynucleotides, including mixtures thereof.

The terms “polynucleotide” and “oligonucleotide” are used interchangeably and refer to a polymeric form of nucleotides of any length, either deoxyribonucleotides or ribonucleotides or analogs thereof. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “difficult to express protein” herein refers to a recombinant protein to be produced at large quantities for therapeutic or industrial applications. In some embodiments, a difficult to express protein has an expression level, from high titer transient mammalian expression kits such as ExpiCHO™, that is <50 mg/L, <100 mg/L, <200 mg/L or <300 mg/L. Difficult to express proteins are produced at levels significantly below the average expression levels of other comparable sized proteins. An exemplary easy to express proteins include antibodies. Difficult to express proteins may have non-natural combinations of protein domains, resulting in low expression, aggregation, and/or misfolding. Further, naturally occurring proteins may also be difficult to express due to exceptionally large sizes, tendency to misfold, tendency to aggregate, reliance on rare co-folding modulators.

Cell “clones” are populations of cells that were isolated from a single cell population via methods including but not limited to single cell printing, limiting dilution, cell colony isolation, and FACS sorting.

“Directed evolution” may refer to a process whereby a population of cells is subjected to a condition that increases the probability of generating a cell population with a specific phenotype that is infrequent or absent in the original cell population. In another instance, directed evolution may refer to strategies to increase or decrease the frequency of a phenotype where a selective condition is not applied. In this case, cell populations are screened for a phenotype, and said phenotype is enriched through any number of cell isolation techniques.

The term “cell engineering” herein refers to strategies to alter a cell via directed evolution or via known direct methods of gene upregulation or downregulation. An “engineered cell” is a cell that has been subject to one or more cell engineering strategies.

### II. Cell Engineering

Protein folding pathways are important for expression of difficult to fold and difficult to express proteins. It has been shown that unmodified prokaryotic and eukaryotic cells are not suited for expression of large and complex recombinant proteins as seen by the exceptionally low expression of an exemplary difficult to express protein, Factor VIII. Commonly reported titers of antibodies can be found up to 5 g/L, however the maximum reported titers of Factor VIII in CHO cells remain at 16 mg/L. This >300 fold difference in expression has been attributed to inefficient protein folding by numerous previous researchers. Inefficient protein folding results in oxidative stress signaling, triggering of the unfolded protein response (UPR), and endoplasmic reticulum stress (ERS) pathways. These pathways lead to cell death and reduced protein transcription and translation rates causing the observed reduced titers for hard to express proteins such as Factor VIII.

It is contemplated that the hard to express protein problem is a result of a bottleneck caused by a lack of appropriate protein folding chaperones in the ER at the correct concentrations to support efficient folding to achieve gram level expression of complex proteins. This has been explored through cell engineering efforts employed to increase the bioavailability of protein folding and secretion chaperones shown to be relevant to recombinant protein production. Overexpression of protein folding chaperones, however, does not yield consistent results between different proteins, different cell lines, and due to clone to clone variation. Cell engineering processes are not even applicable between clones from the same cell line expressing the same protein. Thus, the need for novel, widely applicable cell engineering techniques for expression of difficult to express proteins is great and as yet, unmet.

Previous reports have shown the use of protein folding and secretion chaperone overexpression which is made useful with the help of high throughput screening. Libraries of chaperones may be developed and tested on individual clones and can increase expression of difficult to express proteins. This is a cumbersome, expensive, and time-consuming process that does not have guaranteed results.

The present disclosure provides a new, generalizable solution to the protein folding problem hindering the mass production of potentially therapeutically relevant difficult to express proteins. In one embodiment, the disclosed cell engineering method extends cell longevity, reduces UPR response signaling, and/or increases expression of difficult to express proteins. Not being by bound by any particular theory, it is contemplated that the cell engineering methods described herein (a) reduces reactive oxygen species which can be seen as a key mediator of the unfolded protein response and/or (b) improves turnover of oxidative protein folding chaperones such as PDI and other eukaryotic protein folding chaperones by chemically reducing them to an activated state. In some embodiments, methods of upregulating redox associated enzymes are provided to aid in redox protein folding in eukaryotic cells.

In one embodiment, cells may be engineered to overexpress one or more ROS reducing enzyme. Non-limiting examples of such enzymes include superoxide dismutases, superoxide reductases, catalases, glutathione peroxidases, peroxiredoxins, and those listed in Table I.

In another embodiment, the engineered cells expressing at least one of the previously described dismutases, catalases, peroxidases, or peroxiredoxins are additionally or alternatively engineered to overexpress a reductase, which can work synergistically with a complementary enzyme.

It is contemplated that glutathione-S-transferase enzymes play a previously unexplored and key role in protein folding and ER stress. Thus, in another embodiment of the disclosure, cells are engineered to overexpress at least one glutathione-S-transferase enzyme. Glutathione-S-transferase enzymes include but are not limited to glutathione-S-transferase alpha (GSTA1, GSTA2, GSTA3, GSTA4, GSTA5), delta, kappa (GSTK1), mu (GSTM1, GSTM1L, GSTM2, GSTM3, GSTM4, GSTM5), omega (GSTO1, GSTO2), pi (GSTP1), theta (GSTT1, GSTT2, GSTT4), zeta (GSTZ1), microsomal (MGST1, MGST2, MGST3).

In another embodiment, glutathione reductase may be overexpressed to work synergistically with endogenous, unmodified levels of glutathione-S-transferase or glutathione peroxidase.

In another embodiment, the previously described cell engineered to overexpress a glutathione-S-transferase or glutathione peroxidase enzyme is additionally engineered to overexpress glutathione reductase. Glutathione reductase increases the availability of glutathione such that glutathione-S-transferase and glutathione peroxidase may function appropriately.

TABLE I

| Enzyme Family | Gene Symbol | EC | Enzyme Function |
|---|---|---|---|
| Superoxide | sodN | 1.15.1.1 | Protects cells from oxidative damage by |
| Dismutase | | | catalyzing the disproportionation of the |
| | | | cytotoxic superoxide radical (O- |
| | | | 2) to hydrogen peroxide and molecular |
| | | | oxygen |
| | SOD1 | 1.15.1.1 | Catalyzes the disproportionation of |
| | | | superoxide to hydrogen peroxide and |
| | | | dioxygen |
| | SOD2 | 1.15.1.1 | Binds to the superoxide byproducts of |
| | | | oxidative phosphorylation and converts them |
| | | | to hydrogen peroxide and diatomic oxygen |
| | SOD3 | 1.15.1.1 | Catalyzes the dismutation of two superoxide |
| | | | radicals into hydrogen peroxide and oxygen. |
| | | | Thought to protect tissues from oxidative |
| | | | stress. |
| | CCS | | Delivers copper to SOD1 |
| Superoxide | dfx | 1.15.1.2 | Enzyme that catalyzes the conversion of |
| reductase | | | highly reactive and toxic superoxide (O₂⁻) |
| | | | into less toxic hydrogen peroxide (H₂O₂) |
| Catalase | CAT | 1.11.1.6 | catalyzes the decomposition of hydrogen |
| | | | peroxide to water and oxygen |
| Glutathione | GPX1 | 1.11.1.9 | Functions in the detoxification of hydrogen |
| Peroxidase | | | peroxide, ubiquitously expressed in many |
| | | | tissues, where it protects cells from oxidative |
| | | | stress |
| | GPX2 | 1.11.1.9 | Responsible for the majority of the |
| | | | glutathione-dependent hydrogen peroxide- |
| | | | reducing activity in the epithelium of the |
| | | | gastrointestinal tract |
| | GPX3 | 1.11.1.9 | Functions in the detoxification of hydrogen |
| | | | peroxide, extracellular GPx found in plasma |
| | GPX4 | 1.11.1.9 | Phospholipid hydroperoxidase that protects |
| | | | cells against membrane lipid peroxidation |
| | GPX5 | 1.11.1.9 | Selenium-independent, and has been |
| | | | proposed to play a role in protecting the |
| | | | membranes of spermatozoa from the |
| | | | damaging effects of lipid peroxidation and/or |
| | | | preventing premature acrosome reaction |
| | GPX6 | 1.11.1.9 | Functions in the detoxification of hydrogen |
| | | | peroxide. Expression of this gene is restricted |
| | | | to embryos and adult olfactory epithelium |
| | GPX7 | 1.11.1.9 | Non-selenocysteine containing phospholipid |
| | | | hydroperoxide glutathione peroxidase |
| | | | essential for alleviating oxidative stress in |
| | | | breast cancer cells |
| | GPX8 | 1.11.1.9 | Functions in the detoxification of hydrogen |
| | | | peroxide. Highly expressed in muscle tissue. |
| Peroxiredoxin | PRDX1 | 1.11.1.15 | Member of the peroxiredoxin family of |
| | | | antioxidant enzymes, which reduce hydrogen |
| | | | peroxide and alkyl hydroperoxides. May play |
| | | | an antioxidant protective role in cells, and |
| | | | may contribute to the antiviral activity of |
| | | | CD8(+) T-cells. |
| | PRDX2 | 1.11.1.15 | May play an antioxidant protective role in |
| | | | cells, and may contribute to the antiviral |
| | | | activity of CD8(+) T-cells. This protein may |
| | | | have a proliferative effect and play a role in |
| | | | cancer development or progression. |
| | PRDX3 | 1.11.1.15 | Protein with antioxidant function and is |
| | | | localized in the mitochondrion |
| | PRDX4 | 1.11.1.15 | Antioxidant enzyme of the peroxiredoxin |
| | | | family. The protein is localized to the |
| | | | cytoplasm. |
| | PRDX5 | 1.11.1.15 | Has been shown that PRDX5 can be localized |
| | | | to mitochondria, peroxisomes, the cytosol, |
| | | | and the nucleus. Can use cytosolic or |
| | | | mitochondrial thioredoxins to reduce alkyl |
| | | | hydroperoxides or peroxynitrite |
| | PRDX6 | 1.11.1.15 | Involved in redox regulation of the cell; it can |
| | | | reduce H(2)O(2) and short chain organic, |
| | | | fatty acid, and phospholipid hydroperoxides. |
| | | | It may play a role in the regulation of |
| | | | phospholipid turnover as well as in protection |
| | | | against oxidative injury. |
| Glutathione | GSR | EC 1.8.1.7 | Catalyzes the reduction of glutathione |
| Reductase | | | disulfide (GSSG) to the sulfhydryl form |
| | | | glutathione (GSH). |
| Glutathion-S- | GSTA1, GSTA2, | EC 2.5.1.18 | These enzymes function in the detoxification |
| Transferase | GSTA3, GSTA4, | | of electrophilic compounds, including |
| | GSTA5 | | carcinogens, therapeutic drugs, |
| | | | environmental toxins and products of |
| | | | oxidative stress, by conjugation with |
| | | | glutathione. |
| | GSTK1 | EC 2.5.1.18 | It belongs to the superfamily of enzymes |
| | | | known as glutathione S-transferase (GST), |
| | | | which are mainly known for cellular |
| | | | detoxification. Its structure has been found to |
| | | | be similar to bacterial HCCA (2- |
| | | | hydroxychromene-2-carboxylate) isomerases |
| | | | and bacterial disulphide-bond-forming DsbA |
| | | | oxidoreductase. |
| | GSTM1, GSTM2, | EC 2.5.1.18 | The mu class of enzymes functions in the |
| | GSTM3, GSTM4, | | detoxification of electrophilic compounds, |
| | GSTM5 | | including carcinogens, therapeutic drugs, |
| | | | environmental toxins, and products of |
| | | | oxidative stress, by conjugation with |
| | | | glutathione. |
| | GSTO1, GSTO2 | EC 2.5.1.18 | In mouse, the encoded protein acts as a small |
| | | | stress response protein, likely involved in |
| | | | cellular redox homeostasis. |
| | GSTP1, GSTP2 | EC 2.5.1.18 | The glutathione S-transferase pi gene |
| | | | (GSTP1) is a polymorphic gene encoding |
| | | | active, functionally different GSTP1 variant |
| | | | proteins that are thought to function in |
| | | | xenobiotic metabolism and play a role in |
| | | | susceptibility to cancer, and other diseases. |
| | GSTT1, GSTT2, | EC 2.5.1.18 | GST-theta is a member of a superfamily of |
| | GSTT3, GSTT4 | | proteins that catalyze the conjugation of |
| | | | reduced glutathione to a variety of |
| | | | electrophilic and hydrophobic compounds. |
| | GSTZ1 | EC 2.5.1.18 | GSTZ1 is gene that encodes multifunctional |
| | | | enzymes important in the detoxification of |
| | | | electrophilic molecules, including |
| | | | carcinogens, mutagens, and several |
| | | | therapeutic drugs, by conjugation with |
| | | | glutathione |
| | MGST1, MGST2, | EC 2.5.1.18 | This gene encodes a protein that catalyzes the |
| | MGST3 | | conjugation of glutathione to electrophiles |
| | | | and the reduction of lipid hydroperoxides. |
| | | | This protein is localized to the endoplasmic |
| | | | reticulum and outer mitochondrial membrane |
| | | | where it is thought to protect these |
| | | | membranes from oxidative stress. |

In a preferred aspect of any of the above embodiments, the engineering is achieved by mutating or deleting at least part of the gene for down-regulation or introducing one or more copies of the gene or its coding sequence for up-regulation.

In one embodiment, the previously described engineered cells further include an exogenous coding sequence (“gene of interest” or GOI). The GOI can be included on a separate vector (e.g., plasmid) or integrated to one of the chromosomes of the cell. In one embodiment, the GOI encodes a polypeptide which can be a therapeutic protein. In one embodiment, the GOI encodes an antibody or an antibody fragment.

In one embodiment, the engineered cell is a mammalian cell and preferably a human cell. In one embodiment, the cell is a CHO cell, such as CHO lineage− DG44, DxB11, CHOM (Selexis), CHOs (Life Tech), CHOK1SV (Lonza), or CHOZN (Sigma). In one embodiment, the cell is NSO− mouse, BHK, PerC6, K562, or Cos1&7 cells. In another embodiment, the engineered cell is another eukaryotic organism such as a fungus cell. In one embodiment the fungus cell is Saccharomyces, Pichia, or C1 (Dyadic).

Methods of using any cell of the present disclosure for expressing or producing a product of the GOI are also provided.

### III. Methods for Up- or Down-Regulating a Gene in a Cell

Methods for up-regulating a gene (e.g., increasing the biological activity of the gene) in a cell are known in the art. In one aspect, the gene level is increased by increasing the amount of a polynucleotide encoding gene, as provided above, wherein that polynucleotide is expressed such that new gene is produced. In another aspect, increasing the gene level is accomplished by increasing the transcription of a polynucleotide encoding gene, or alternatively translation of gene, or alternatively post-translational modification, activation or appropriate folding of gene. In yet another aspect, increasing gene level is increased by increasing the binding of the protein to appropriate cofactor, receptor, activator, ligand, or any molecule that is involved in the protein's biological functioning. In some embodiments, increasing the binding of gene to the appropriate molecule is increasing the amount of the molecule. In one aspect of the embodiments, the molecule is a polypeptide. In another aspect of the embodiments, the molecule is a small molecule. In a further aspect of the embodiments, the molecule is a polynucleotide. In one embodiment, the molecule is a combination of any of a small molecule, a polypeptide, or a nucleic acid. In a further aspect of the embodiment, the molecule is a CRISPR guided activator.

Methods of increasing the amount of polynucleotide in a cell are known in the art and can be modified for increasing the amount of a polynucleotide encoding gene. In one aspect, the polynucleotide can be introduced to the cell and expressed by a gene delivery vehicle that can include a suitable expression vector.

Suitable expression vectors are well-known in the art, and include vectors capable of expressing a polynucleotide operatively linked to a regulatory element, such as a promoter region and/or an enhancer that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

Non-viral vector may include plasmid that comprises a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

Methods of down-regulating a gene (e.g., decreasing the biological activity or inhibiting a gene product) are known in the art. Non-limiting examples include mutating the gene, deleting part or whole of the sequence of the gene, or inhibiting the gene with siRNA, dsRNA, miRNA, antisense polynucleotide, ribozymes, triplex polynecleotide, antibody, or an antibody variant.

“Short interfering RNAs” (siRNA) refer to double-stranded RNA molecules (dsRNA), generally, from about 10 to about 30 nucleotides in length that are capable of mediating RNA interference (RNAi). “RNA interference” (RNAi) refers to sequence-specific or gene specific suppression of gene expression (protein synthesis) that is mediated by short interfering RNA (siRNA). As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA. A siRNA may be chemically modified to increase its stability and safety.

“Double stranded RNAs” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

“MicroRNAs” (miRNA) refer to single-stranded RNA molecules of 18-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

### EXAMPLES

The disclosure is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims

### Example 1

### Directed Evolution of Cells for Increased GST Expression

In one example, CHO cells were engineered to overexpress oxidative stress related genes through methods of directed evolution. The starting pool of cells were compared against the evolved pool by RNA microarray analysis. The evolved pool of CHO cells will be referred to as “SUPERCELL™” and the original pool of CHO cells will be referred to as “CHOK1” or “CHO”. Briefly, at multiple representative time points, 1E6 cells were saved for RNA microarray analysis. A total of 4 different time points were utilized for the CHOK1 and SUPERCELL™. The samples were prepared and loaded onto a CHO Gene ST Affymetrix Microarray, and hybridization quality was confirmed. Comparative signal intensity was used after internal normalization to compare relative expression of different glutathione related genes across the parental and subcloned population.

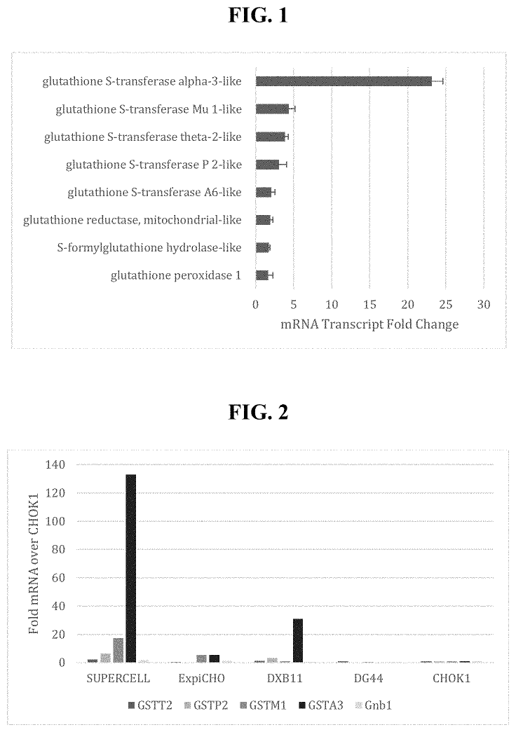

The relevant results are summarized in FIG. 1. Shown are the relative fold increases in different mRNA transcript levels with an n=4 for each of the SUPERCELL™ samples and CHOK1 samples. The directed evolution process resulted in a general upregulation of glutathione related genes. Of particular interest is the 23-fold upregulation of GSTA3, as well as the potentially synergistically interacting glutathione related enzymes that are not glutathione-S-transferases. These include S-formylglutathione hydrolase, glutathione peroxidase 1, and glutathione reductase.

The results of the experiment were further confirmed by qPCR and additional cell lines commonly used in bioproduction were assayed for a subset of glutathione-S-transferases. The results shown in FIG. 2 confirms the unique upregulation of GST enzymes in SUPERCELL™ in comparison to all other tested CHO cell lines.

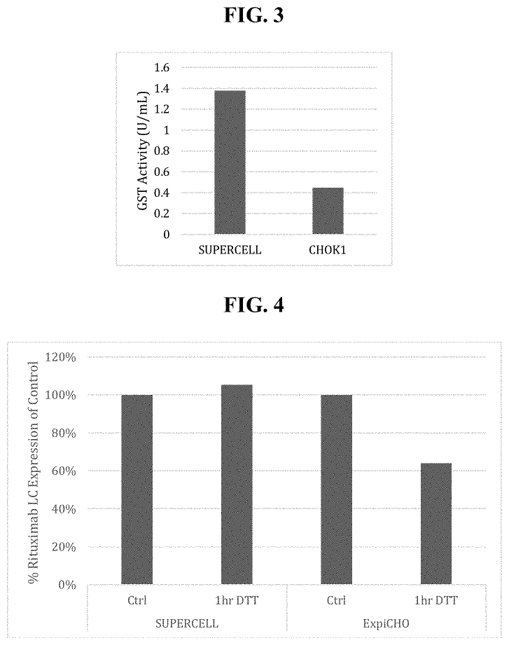

A GST activity assay was further performed to confirm the qPCR and mRNA microarray data correlates to higher general GST activity. Briefly, cells were counted by Vicell, 4M cells for SUPERCELL™ and CHOK1 were lysed in 2 mM EDTA+0.1M Phosphate, clarified lysate was isolated, and used diluted and undiluted as specified in “QuantiChrom™ Glutathione S-transferase Assay Kit” cat no. DGST-100 to determine Glutathione S-transferase activity in CHOK1 and SUPERCELL™. The results shown in FIG. 3 support the microarray and qPCR data showing increased GST transcript levels.

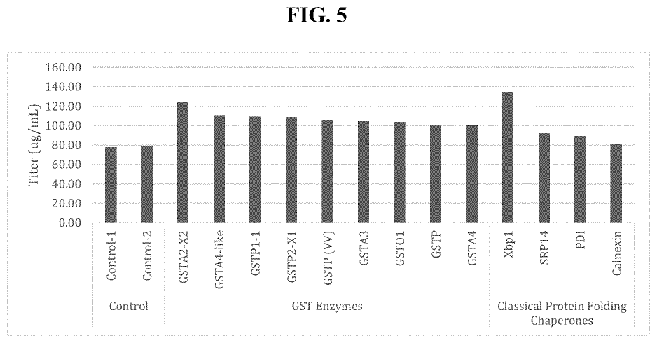

The SUPERCELL™ cell line is postulated to be able to handle higher levels unfolded protein response and oxidative stress before activating XBP1 splicing and EIF2a phosphorylation, shutting down protein expression and reducing recombinant protein expression levels. To test this, we transfected ExpiCHO and SUPERCELL™ using the Neon transfection system with plasmid expressing Rituximab LC. 24 hours after transfection, we treated the cells with 1mM DTT. The cells were transferred to fresh media after 1 hour and cultured to produce protein for 7 days before harvesting the supernatant and quantifying the resulting produced protein by Octet Protein L binding. The results are shown in FIG. 4. The results show that in comparison to the cells not treated with DTT, there is no difference in productivity in the SUPERCELL™ cell line. However, the ExpiCHO cell line titers drop by ˜40% with DTT treatment. We expect that this resilience to artificially induced UPR will aid the SUPERCELL™ cell line in production of difficult to express proteins where UPR may be induced naturally due to high expression of aggregated proteins.

In an embodiment of the present invention, GST enzymes may be increased in expression through the implementation of a directed evolution approach to selecting for cells resistant to UPR induced stress followed by assessment of cell qualities by microarray, mRNA sequencing, qPCR, GST activity assay, protein expression assays, or other FACS or plate-based assays to analyze intracellular GST content or activity or redox potential. Further, in an embodiment of the precent invention, after selecting a population of cells displaying increased GST activity or increased protein expression, the cell line may then be further modified to express a recombinant protein of interest by (a) transiently transfecting a first expression cassette comprising a recombinant protein of interest, a promoter, and a polyadenylation signal such that the expression cassette yields higher productivities of recombinant protein than a control cell line which has not been modified and screened for increased GST activity (b) stably integrating a first expression cassette comprising a recombinant protein of interest such that the first expression cassette yields higher productivities of recombinant protein than a control cell line which does not has not been modified and screened for increased GST activity.

### Example 2

### Overexpressing GST Enzymes in Cells

To confirm that recombinant overexpression of GST enzymes in mammalian cells is a viable method to improve protein expression levels, we transiently cotransfected a series of GST enzymes along with Tysabri HC and Tysabri LC expression plasmids in the ExpiCHO system. In parallel we also tested the cotransfection of classical protein folding chaperones with Tysabri HC and Tysabri LC. The cells were cultured for 12 days in 10 mL spin tubes. The harvest supernatant was analyzed by Octet Protein A binding for recombinant protein expression levels. The results are shown in FIG. 5. As expected, an increase in protein expression levels can be seen with all tested GST enzymes. Surprisingly, GST overexpression outperformed classical protein folding chaperones described elsewhere including SRP14 (signal recognition particle 14), PDI (Protein disulfide-isomerase), and Calnexin.

In a preferred embodiment of the present invention, a GST enzymes may be stably integrated into a cell line expressing a recombinant protein by (1) transfecting a cell with an expression vector containing a first promoter, a GST enzyme, a first polyadenylation site as well as a second promoter, a resistance marker, and a second polyadenylation site, (2) selecting the cells with a selection marker corresponding to the resistance marker, (3) recovering the cells and testing by any of the previously described methods for improved GST activity or improved protein expression, (4) and finally (a) transiently transfecting a third expression cassette comprising a recombinant protein of interest and producing recombinant protein through transient expression (b) stably integrating a third expression cassette comprising a recombinant protein of interest such that the third expression cassette yields higher productivities of recombinant protein than a control cell line which does not comprise upregulated GST enzymes.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A modified eukaryotic cell comprising;
a. one or more epigenetic or genetic alterations resulting in increased expression of a gene described in Table I, and
b. an exogenous polynucleotide sequence comprising an expression cassette comprising a first promoter, a heterologous polynucleotide, a polyadenylation sequence.

2. The cell of claim 1, wherein the genetic or epigenetic alteration is recombinant overexpression or CRISPR-guided activation (CRISPRa) of the gene.

3. The cell of claim 2, wherein the gene is a glutathione-s-transferase enzyme.

4. The cell of claim 3, wherein the glutathione-S-transferase enzyme is selected from the group consisting of:
a. GSTA3,
b. GSTA4,
c. GSTA6,
d. GSTT2,
e. GSTM1,
f. GSTM7, and
g. GSTP2.

5. The cell of claim 3, further comprising a second genetic or epigenetic alteration resulting in enhanced activity of a second gene from Table I.

6. The cell of claim 5, wherein the second genetic or epigenetic alteration is recombinant overexpression or CRISPR-guided activation (CRISPRa) of the second gene.

7. The cell of claim 6, wherein the second gene is a superoxide dismutase, superoxide reductase, catalase or peroxiredoxin.

8. The cell of claim 6, wherein the second gene is a glutathione reductase or glutathione peroxidase.

9. The cell of claim 7, wherein the second gene is selected from the group consisting of:
a. GPX1,
b. GPX7, and
c. GPX4.

10. The cell of claim 8, wherein the gene is glutathione reductase.

11. The cell of claim 6, wherein the gene and the second gene are, respectively,
a. GSTA1 and Glutathione reductase,
b. GSTA2 and Glutathione reductase,
c. GSTA3 and Glutathione reductase,
d. GSTA4 and Glutathione reductase,
e. GSTA5 and Glutathione reductase,
f. GSTA6 and Glutathione reductase,
g. GSTM1 and Glutathione reductase,
h. GSTM7 and Glutathione reductase,
i. GSTP1 and Glutathione reductase,
j. GSTP2 and Glutathione reductase,
k. GSTT1 and Glutathione reductase,
l. GSTT2 and Glutathione reductase,
m. GSTT3 and Glutathione reductase, or
n. GSTT4 and Glutathione reductase

12. The cell claim 1, wherein the heterologous polynucleotide encodes a therapeutic protein.

13. The cell of claim 12, wherein the cell is a Chinese Hamster Ovary cell.
